(19) United States
(12) Patent Application Publication
(10) Pub. No.: US 2025/0049424 A1
(43) Pub. Date: Feb. 13, 2025

(54) METHOD AND APPARATUS FOR EPICARDIAL ACCESS

(71) Applicant: CIRCA SCIENTIFIC, INC., ENGLEWOOD, CO (US)

(72) Inventor: DAVID WILLENBRINK, DENVER, CO (US)

(73) Assignee: CIRCA SCIENTIFIC, INC., ENGLEWOOD, CO (US)

(21) Appl. No.: 18/798,758

(22) Filed: Aug. 8, 2024

Related U.S. Application Data
(60) Provisional application No. 63/531,480, filed on Aug. 8, 2023.
(60) Provisional application No. 63/659,250, filed on Jun. 12, 2024.

(51) Int. Cl. A61B 17/00; A61B 17/34; A61B 90/00; A61B 90/30

(52) U.S. Cl. CPC A61B 17/00234; A61B 17/3417; A61B 90/30; A61B 90/361; A61B 2017/00026; A61B 2017/00238; A61B 2017/00305; A61B 2017/00323; A61B 2017/00367; A61B 2017/00477; A61B 2017/00557; A61B 2017/00907; A61B 2090/306

(57) ABSTRACT

A medical device comprising a handle comprising a first actuator coupled to a first set of articulation cables; and a second actuator coupled. The device may include a shaft comprising a distal articulation section configured to bend with a distal articulation deflection angle and a distal articulation radius, wherein actuation of the first articulation member is configured to induce the distal articulation deflection angle in the distal articulation section. The device may further include a balloon configurable in a deflated state and an inflated state.

### CROSS-REFERENCE TO RELATED APPLICATIONS

The present application claims the benefit of U.S. Patent Application No. 63/659,250 for METHOD AND APPARATUS FOR EPICARDIAL ACCESS, filed Jun. 12, 2024, and U.S. Patent Application No. 63/531,480 for METHOD AND APPARATUS FOR EPICARDIAL ACCESS, filed Aug. 8, 2023, the entire contents of which are incorporated herein by reference in their entirety.

### FIELD OF THE INVENTION

The present disclosure relates to a medical device. More specifically, the present disclosure relates to a method and apparatus for use in accessing the epicardium and/or treating cardiac conditions.

### INTRODUCTION

Throughout many cardiac surgeries or medical interventions, visualization and guidance of instruments, such as catheters, may be limited. It may be challenging to identify various tissues and bodily structures, e.g., vessels, scar tissue, fat pads, and nerves, which can cause unsought complications.

Throughout many cardiac surgeries or medical interventions, control, and guidance of instruments, such as catheters, may be limited. It is necessary for catheters, and other introducers, to provide a degree of flexibility to maneuver through the vasculature of a patient to perform medical procedures. A physician or practitioner controls the catheter through a handle located outside the body to manipulate the catheter as it is inserted and advanced through the patient. Given the large number of uses, there is a need for various handles to accommodate preferences.

It would be desirable, therefore, to provide methods and apparatuses to overcome these and other deficiencies.

### SUMMARY OF THE INVENTION

This Summary is provided to introduce a selection of concepts in a simplified form that are further described below in the Detailed Description. This Summary is not intended to identify key features or essential features, nor is it intended to limit the scope of the claims included herewith.

An aspect of the present disclosure may include a medical device comprising a handle configured to be gripped by a medical professional to steer and insert a shaft into a cavity of a patient.

The cavity may include, but is not limited to, a vessel, a canal, a tissue opening, or other opening within the body of the patient. In order to advance the shaft into the cavity of a patient, including, but not limited to the vascular system, it is contemplated that the shaft must be substantially stiff to move through the vasculature of a patient. That is, while gripping the shaft on its proximal end, the distal end must have sufficient stiffness to not kink while pushing the proximal end of the guide tube when the distal end encounters resistance. Prior art stiff catheters and guide tubes, however, create patient discomfort and potentially dangerous situations when trying to advance a catheter through circuitous portions of the vasculature.

It is contemplated that the operator holds the handle while advancing and steering the shaft through the vasculature of the patient. Advantageously, the medical practitioner can flex (i.e., deflect or bend) the distal end of the guide tube in the same plane of deflection with either hand or both hands employing either a first actuator, a second actuator, or both actuators at the same time. This enhances the practitioner's ability to effectively steer the shaft in the patient by optimizing the operator's steering options to steer the distal end of the guide tube.

An aspect of the present disclosure may include a medical device comprising a handle, a shaft coupled to the handle, and a balloon. The handle may comprise a first actuator mechanically coupled to a first set of articulation cables, and a second actuator. The shaft may comprise a distal articulation section configured to bend with a distal articulation deflection angle and a distal articulation radius. The distal articulation section may be mechanically coupled to the first set of articulation cables, and actuation of the first actuator may be configured to induce the distal articulation deflection angle in the distal articulation section. The balloon may be disposed of adjacent to the distal articulation section of the shaft. The balloon may be configurable in a deflated state and an inflated state.

An aspect of the present disclosure may include the shaft further comprising a proximal articulation section configured to bend with a proximal articulation deflection angle and a proximal articulation radius. The proximal articulation section may be mechanically coupled to a second set of articulation cables. In one embodiment, actuation of the proximal articulation control knob may induce the proximal articulation deflection angle in the proximal articulation section.

An aspect of the present disclosure may further comprise a camera disposed on the shaft.

An aspect of the present disclosure may further comprise a light fiber bundle disposed in the shaft, traversing from at least the handle to the distal articulation section.

An aspect of the present disclosure may further comprise a light source disposed within the distal articulation section.

In accordance with an aspect of the present disclosure, actuation of the first actuator may bend the shaft in a first plane, and actuation of the second actuator may bend the shaft in a second plane. In one embodiment, the first plane and the second plane may be perpendicular to another.

In accordance with one aspect of the present disclosure, the balloon may be asymmetric around the shaft. In accordance with an aspect of the present disclosure, the balloon may be symmetric around the shaft.

An aspect of the present disclosure may further comprise one or more impedance sensors disposed of at the distal end portion of the flexible shaft. In an embodiment, any of the one or more impedance sensors may be disposed distally to the balloon. In one embodiment, any of the one or more impedance sensors may be disposed proximally to the balloon. The one or more impedance sensors may be configured to permit anatomical mapping and, more specifically, to map the location of the shaft within the patient.

An aspect of the present disclosure may include the handle further comprising a first articulation lock in mechanical communication with the first actuator, the first articulation lock configured to lock the distal articulation section in a desired position.

An aspect of the present disclosure may include the handle further comprising one or more proximal articulation drive nuts in mechanical communication with a second set of articulation cables. In some embodiments, the handle may further comprise one or more proximal articulation pulleys in mechanical communication with the second set of articulation cables.

In accordance with one aspect of the present disclosure, the first and second actuators may be coupled to a first drive adapted to pull on the first set of articulation cables and a spindle adapted to pull on a second set of articulation cables, wherein the first drive and the spindle are disposed about a linear drive shaft.

An aspect of the present disclosure may comprise a disc in frictional contact with the first actuator to prevent the first actuator from rotating without manipulation.

Other features and advantages will become apparent to those skilled in the art upon review of the following drawings, the detailed description, and the appended claims.

### BRIEF DESCRIPTION OF THE DRAWINGS

Objects, aspects, features, and advantages of embodiments disclosed herein will become more fully apparent from the following detailed description, the appended claims, and the accompanying drawing figures in which like reference numerals identify similar or identical elements. Reference numerals that are introduced in the specification in association with a drawing figure may be repeated in one or more subsequent figures without additional description in the specification in order to provide context for other features, and not every element may be labeled in every figure. The drawing figures are not necessarily to scale, emphasis instead being placed upon illustrating embodiments, principles and concepts. The drawings are not intended to limit the scope of the claims included herewith.

FIG. 1 is an illustration of an embodiment of the device.

FIG. 2 shows an embodiment of a handle of the device.

FIG. 3 is a sectional view of an embodiment of the handle of the device.

FIG. 4 is an exploded view of an embodiment of the handle of the device.

FIG. 5 shows an embodiment of a shaft tip of the device.

FIG. 6 shows an embodiment of a shaft tip of the device.

FIG. 7 is an illustration of the device exhibiting bidirectional steering.

FIG. 8 shows an embodiment of shaft tip deflection.

FIG. 9A is an illustration of customizable shaft tip deflection.

FIG. 9B is an illustration of customizable shaft tip deflection.

FIG. 10 shows an embodiment of the device for use with a medical instrument.

FIG. 11 is an illustration of an embodiment of the device.

FIG. 12 is an illustration of an embodiment of the device.

FIG. 13 is an illustration of one embodiment of a handle of the device.

FIG. 14 shows an embodiment of a handle of the device.

FIG. 15 is a sectional view of an embodiment of the handle of the device.

As used herein, the term “tensioning line” is used to describe any number of devices or mechanisms by which a force is applied to a portion of a shaft. The force may be applied as a result of a “pulling” force exerted on a connector or wire extending from the distal end of the shaft to a proximal end of the shaft, a “pushing” force, the result of deformation of a connector or wire in the distal end of the shaft due to a change in temperature, etc. or any other means whereby a flexible element disposed within the distal end of the shaft exerts a flexing or tensioning force on portion of the shaft.

The terms “shaft” or “catheter” are used herein to describe any number of guiding elements used to place a “treating catheter” or “treating instrument” into a patient. In one aspect, the guide catheter is removed before treatment of the patient begins and the treating catheter remains in the patient. The catheter may be a solid guide wire over which the treating catheter is placed or a hollow shaft through which the treating catheter is placed. In one aspect, after placement of the treating instrument, the guide catheter is removed from the patient. In another aspect, however, the guide catheter remains in place while a treating instrument is advanced to a location within the patient and remains in place while one or more treating instruments (e.g., ablation tool, suturing tool, etc.) are employed by a clinician. At the termination of the procedure, the treating instruments and guide catheter are removed from the patient. While specific mention is made herein for use of the technology in the vasculature of the patient, it is understood that the technology may be employed to advance a guide catheter into any portion of the body.

### DETAILED DESCRIPTION

For this disclosure, singular words should be construed to include their plural meaning, unless explicitly stated otherwise. Additionally, the term “including” is not limiting. Further, “or” is equivalent to “and/or,” unless explicitly stated otherwise. Although ranges may be stated as preferred, unless stated explicitly, there may exist embodiments that operate outside of preferred ranges.

In the following detailed description, reference will be made to the accompanying drawing(s), in which identical functional elements are designated with like numerals. The aforementioned accompanying drawings show by way of illustration, and not by way of limitation, specific aspects, and implementations consistent with principles of this disclosure. These implementations are described in sufficient detail to enable those skilled in the art to practice the disclosure and it is to be understood that other implementations may be utilized and that structural changes and/or substitutions of various elements may be made without departing from the scope and spirit of this disclosure. The following detailed description is, therefore, not to be construed in a limited sense.

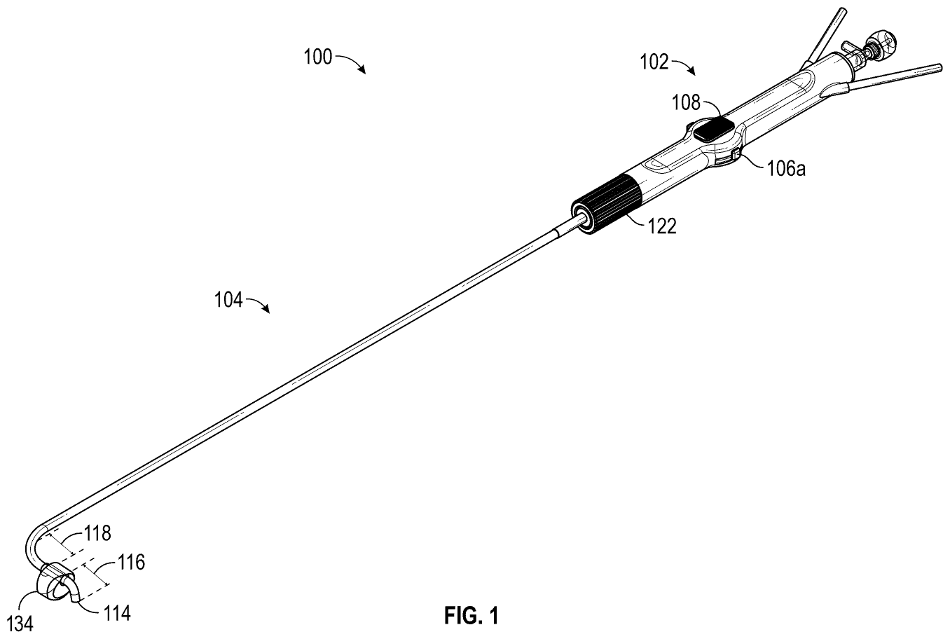

The present disclosure relates to an apparatus for providing navigation within the pericardium and/or providing access to the outside of the pericardium (“tunneling”) while allowing the user to visualize the procedure from the distal end of the device to see what tissues the device is passing through in order to avoid injury to the patient. Referring to FIGS. 1-10, the device 100 may be composed of two major components, such as a handle 102 and a shaft 104. The handle 102 may be adapted for digital manipulation by the user, wherein such manipulation induces one or more actions in the shaft 104. The device 100 may be utilized in conjunction with various medical instruments (i.e., an ablation catheter). Thus, the device 100 may be adapted with various inputs configured to accept fluids or tools.

The shaft 104 may comprise a proximal articulation section 118 and a distal articulation section 116 wherein the digital manipulation of the handle 102 results in articulation of the proximal articulation section 118 and/or the distal articulation section 116.

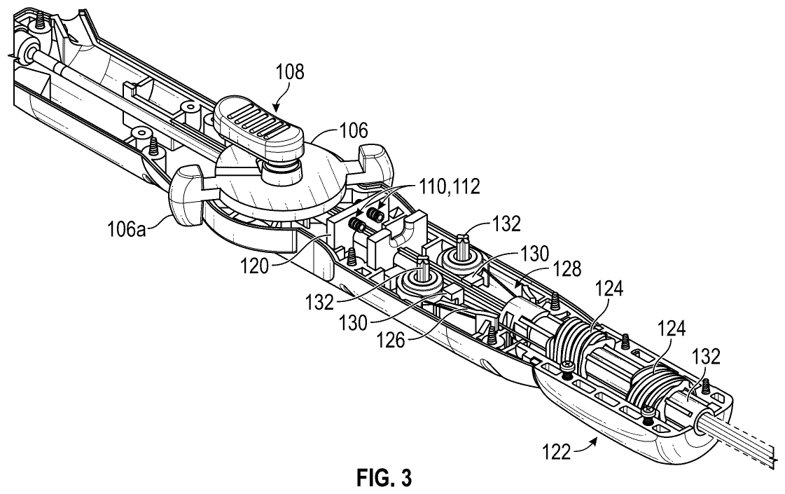

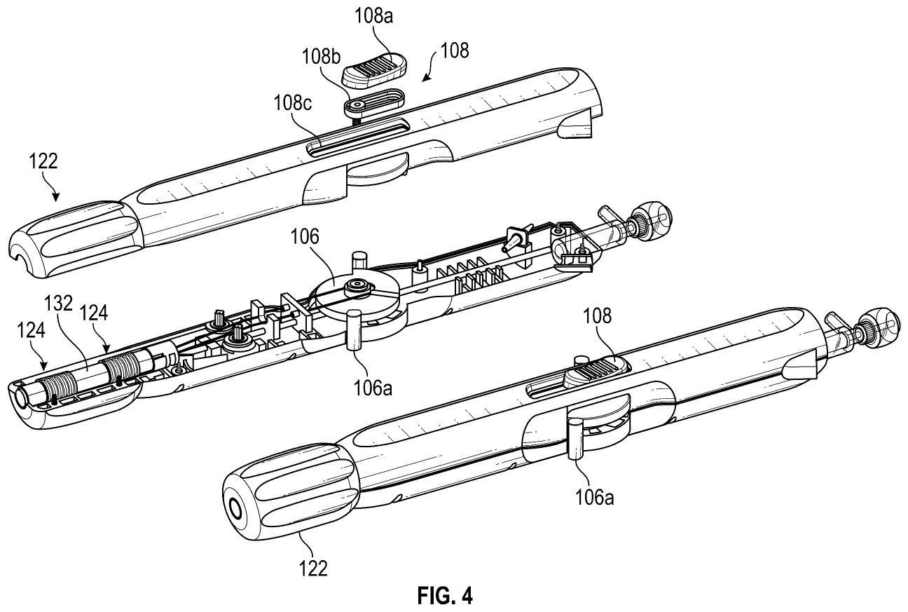

The handle 102 may comprise a first actuator in mechanical communication with the shaft 104, permitting regulation of a selectable angle of the shaft tip. In one embodiment, as illustrated in FIGS. 3-4, the first actuator may comprise a lever 106a and a distal articulation spindle 106. For example, the lever 106a may permit regulation of the distal articulation section 116 of the shaft 104. The lever 106a may be coupled or otherwise in mechanical communication with a distal articulation spindle 106. Accordingly, a wire (or other similar means) may be coupled to the distal articulation spindle 106, such that actuation of the lever 106a induces rotation in the distal articulation spindle 106, further causing the distal articulation spindle 106 to retract the wire, manifesting a bend in the distal articulation section 116. For the purposes of this disclosure, the shaft tip 114 may refer to the segment of shaft 104 ranging from the proximal end of the distal articulation section 116 to the most distal end of the shaft 104. While reference is made herein to the lever 106a and a distal articulation spindle 106, the first actuator may be any actuator.

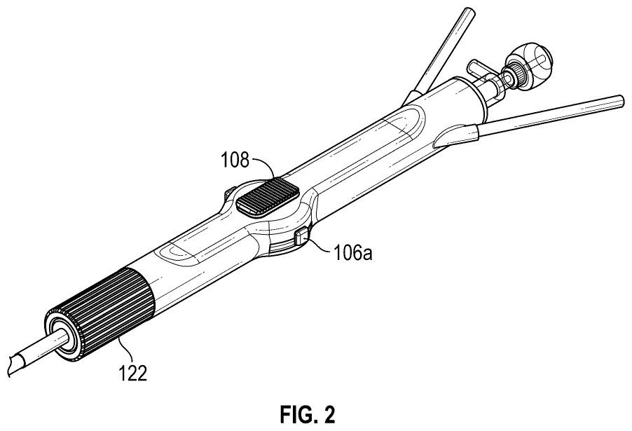

As illustrated in FIG. 2, the handle 102 may include a first articulation lock 108, wherein the first articulation lock 108 is configured to lock a desired angle in the shaft tip 114 (i.e., the distal articulation section 116) by inducing a force against the distal articulation spindle 106. This force may prevent the distal articulation spindle 106 from being permitted to rotate, thus preventing the changes in the wire.

FIG. 4 illustrates an exploded view of one embodiment of the first articulation lock 108. The first articulation lock 108 comprises a channel 108c that securely couples to a slider 108b able to move along the channel 108c. The slider 108b may move from a first, unlocked, position in the channel 108c to a second, locked, position in the channel 108c. The slider 108b may couple to a slider interface 108a, wherein the slider interface 108a comprises a textured surface, for example comprising a plurality of ridges, that increase friction on the slider interface 108a. This may increase the friction at the slider interface 108a of the first articulation lock 108, permitting the operator to easily move the slider 108b from the first to the second position, or vice versa. Of course, other articulation locks may be utilized and the aforementioned is provided as a non-limiting example only.

As illustrated in FIG. 3, the handle 102 may further comprise a first set of articulation cables, illustrated as one or more distal articulation cables 110, and/or distal articulation cable housings 112. In an embodiment, the distal articulation cable housings 112 may be a stacked spring (e.g., tightly wound wire) configured to accept one or more cables, such as the one or more distal articulation cables 110. However, the distal articulation cable housings 112 may be any suitable structure sized to accept the one or more distal articulation cables 110. Accordingly, the distal articulation cables 110 may be coupled to the distal articulation spindle 106, wherein the distal articulation cables 110 are further coupled with a distal articulation section 116 of the shaft tip 114. The handle 102 may also include a distal articulation tension adjustment 120. In an embodiment, the distal articulation tension adjustment 120 may be configured for an initial adjustment (e.g., during manufacture). As a non-limiting example, the distal articulation tension adjustment 120 may be configured to adjust the tension of the articulation cables (e.g., the one or more distal articulation cables 110) such that those pulling in opposite directions are equally tensioned for stability and case of use.

In a further embodiment, the handle 102 may include a second actuator, illustrated as a proximal articulation control knob 122. The proximal articulation control knob 122 may be mechanically coupled to a plurality of proximal articulation drive nuts 124, wherein the proximal articulation drive nuts 124 may be actuated by rotation of the proximal articulation control knob 122. In an embodiment, as shown in FIGS. 1-4, the proximal articulation control knob 122 may be configured to rotate around the longitudinal axis of the shaft 104 and handle 102. Further, a second set of articulation cables, illustrated as one or more proximal articulation cables 126 and/or proximal articulation cable housings 128 may be coupled to the proximal articulation drive nuts 124. The proximal articulation cables 126 and/or proximal articulation cable housings 128 may interface with proximal articulation tension adjustment 130. For the purposes of this disclosure, the proximal articulation tension adjustment 130 may include similar characteristics to that of the distal articulation tension adjustment 120. Further, the proximal articulation cables 126 and/or cable housings 128 may interface with the proximal articulation pulleys 132. The proximal articulation pulleys 132 may be disposed in the handle 102, between the distal articulation spindle 106 and the proximal articulation control knob 122. Thus, the proximal articulation cables 126 may at least partially surround each of the two proximal articulation pulleys 132. In an embodiment, the handle 102 may include two proximal articulation pulleys 132, wherein both pulleys 132 are disposed adjacent to one another, each pulley 132 the same distance from the proximal articulation control knob 122. However, any number of pulleys in various configurations are contemplated.

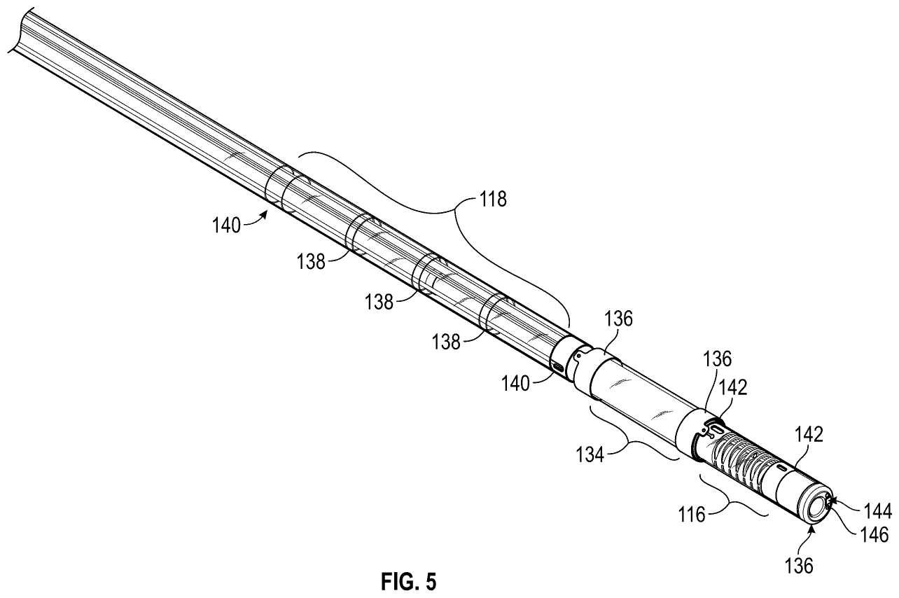

Referring to FIG. 5, the shaft tip 114 may include a plurality of sections or segments. As shown in FIG. 5, the shaft tip 114 may include the proximal articulation section 118 and the distal articulation section 116. In such an embodiment, the proximal articulation section 118 may be manipulated via the proximal articulation control knob 122 and the distal articulation section 116 may be manipulated via the distal articulation spindle 106. For example, rotation of the proximal articulation control knob 122 may increase tension on a first of the one or more proximal articulation cables 126 while decreasing tension on a second of the one or more proximal articulation cables 126, or vice versa.

Further, the shaft tip 114 may include a balloon 134. The distal articulation section 116 may be of greater length than the proximal articulation section 118. However, in various embodiments, the distal articulation section 116 and the proximal articulation section 118 may be configured to any suitable dimensions.

The shaft tip 114 may include one or more impedance sensors 136. In an embodiment, the one or more impedance sensors 136 may be disposed of near a distal end portion of the shaft 104. For example, a first impedance sensor may be disposed between the proximal articulation section 118 and the balloon 134; a second impedance sensor may be disposed between the balloon 134 and the distal articulation section 116; and a third impedance sensor may be disposed on the most distal point of the shaft tip 114. In an embodiment, the one or more impedance sensors 136 may be exposed metal contacts coupled to insulated electrical wires, further coupled to an electroanatomic mapping (EAM) system to provide electrical continuity. For the purposes of this disclosure, the most distal point of the shaft tip 114 may be referred to herein as the “muzzle.” The muzzle may encompass the distal face of the exposed portion of the sidewall of the shaft 104. The muzzle may be rounded or otherwise sized to reduce drag or potential harm to bodily structures.

The shaft 104 may include one or more cable guide loops 138 disposed within the shaft lumen. The one or more cable guide loops 138 may surround and/or partially surround the proximal articulation cables 126 and/or the distal articulations cables 110. For example, the cable guide loops 138 may maintain the position of the proximal articulation cables 126 and/or the distal articulation cables 110, while permitting tensioning of said cables 138/126.

The shaft tip 114 may further comprise one or more proximal cable housing termination points 140. For example, the one or more proximal cable housing termination points 140 may be a proximal weld point. However, any manner of securing the cable housing to the shaft 104 is contemplated and the aforementioned is provided as a non-limiting example only. In one embodiment, a first proximal cable housing termination point may be disposed on a proximal end of the proximal articulation section 118, and a second proximal cable housing termination point may be disposed on a distal end of the proximal articulation section 118. Accordingly, one or more proximal articulation cables 126 may be coupled to one or more of the proximal cable housing termination points 140. Similarly, the shaft tip 114 may comprise one or more distal cable termination points 142. In an embodiment, the one or more distal cable termination points 142 may correspond to distal weld points wherein the termination end of any of the one or more distal articulation cables 110 is secured, for example, via welds, to the shaft 104. In one embodiment, a first distal cable termination point may be disposed on a proximal end of the distal articulation section 116, and a second distal cable termination point may be disposed on a distal end of the distal articulation section 116. Accordingly, one or more distal articulation cables 110 may be coupled to one or more of the distal cable termination points 142. As a non-limiting example, the one or more proximal cable housing termination points 140 may offer an anchor to the one or more proximal articulation cable 126 as to permit tensioning of the one or more proximal articulation cable 126, and thus, deflection of the proximal articulation section 118. Likewise, the one or more distal cable termination points 142 may offer an anchor to the one or more distal articulation cables 110 as to permit the tensioning of the one or more distal articulation cables 110, and thus, deflection of the distal articulation section 116.

In an embodiment, any of the one or more proximal cable housing termination points 140 and the one or more distal cable termination points 142 may be fabricated via a small window in the sidewall of the shaft 104, through which the distal and/or proximal articulation cable housings 112,128 are visible. For example, a small welder may be inserted in such a window to create weld points about the distal and proximal articulation cable housings 112, 128 to define the one or more proximal cable housing termination points 140 and the one or more distal cable termination points 142. However, any means for creating the termination points about the cable housings is contemplated.

In an embodiment, the shaft tip 114 may include a camera 144 (e.g., a Complementary Metal Oxide Semiconductor sensor camera). The camera 144 may be in communication with a computerized device, such that the camera 144 may capture images from the shaft tip 114 and transmit such images to the computerized device. For example, the camera 144 may be in a wired or wireless communication with the computerized device. In some embodiments, the shaft tip 114 may also include a light fiber bundle 146. The light fiber bundle 146 may be one or more fibers traversing any of the length of the shaft 104. Accordingly, the light fiber bundle 146 may encounter one or more light sources within the handle 102 and/or proximate to the handle 102. In such an embodiment, the light fiber bundle 146 may transmit such light to the distal articulation section 116 and/or muzzle of the device 100. For example, the light fiber bundle 146 may be adapted to provide the light necessary to capture suitable images with the camera 144.

The one or more impedance sensors 136 may be disposed within the shaft tip 114. For example, a first impedance sensor may be disposed immediately proximal to the balloon 134 and a second impedance sensor may be disposed immediately distal to the balloon 134. As a non-limiting example, the impedance sensors 136 may be positional markers of the type used in anatomic mapping (e.g., impedance electrodes used in impedance-based anatomical mapping, etc.) and/or may be positioned across an area occupied by the monitoring element. Further, the impedance sensors 136 may be used to three-dimensionally map the location of the shaft tip 114 within the patient's body. The device 100 may be configured for navigating inside the pericardium, wherein the device 100 may also include exposed electrodes along the length of the shaft 104 to allow integration into cardiac impedance mapping systems. Such an embodiment allows the location and orientation of the device 100 path to be illustrated on a 3D model of the heart on an accompanying display. Of course, the impedance sensors may be positioned elsewhere or may be utilized for another purpose and the aforementioned embodiments are described as non-limiting examples only.

Referring to FIG. 7, the balloon 134 may be an offset balloon. Further, the balloon 134 may be configurable in a deflated and an inflated state. The balloon 134 may be inflated to create space between tissue, vessel, or other anatomical features and the shaft tip 114. The space created by the offset balloon 134 in the inflated state may be utilized in bending the shaft tip 114 to create a customizable angle of the shaft tip 114. For example, the inflated balloon 134 may be positioned against tissue, vessel, or other anatomical features, wherein the balloon 134 is configured to provide stability to the shaft tip 114. In another non-limiting example, the balloon 134 may be configured and/or utilized to push anatomical structures away from the shaft tip 114 for the protection of said anatomical structures. In one embodiment, the balloon 134 may be inflated with a liquid and/or a gas. As non-limiting example, the balloon 134 may be inflated with air, saline, water, a saline-water mixture, a contrast agent, and/or a combination thereof. The balloon 134 chamber may be in fluid communication with a fluid source, for example, exterior to the shaft 104 and handle 102. In an embodiment, a fluid lumen may be disposed along the handle 102 and/or the shaft 104, such that fluid may be introduced to and/or removed from the balloon 134. For example, a separate inflation tubing may run parallel within the working channel along the inside of the shaft 104. Thus, in one embodiment, the fluid lumen may be a singular lumen configured to both introduce and remove fluid. In another embodiment, the fluid lumen may be a multi-lumen, comprising two or more lumens, each of the two or more lumens configured for exclusive removal or introduction of fluid.

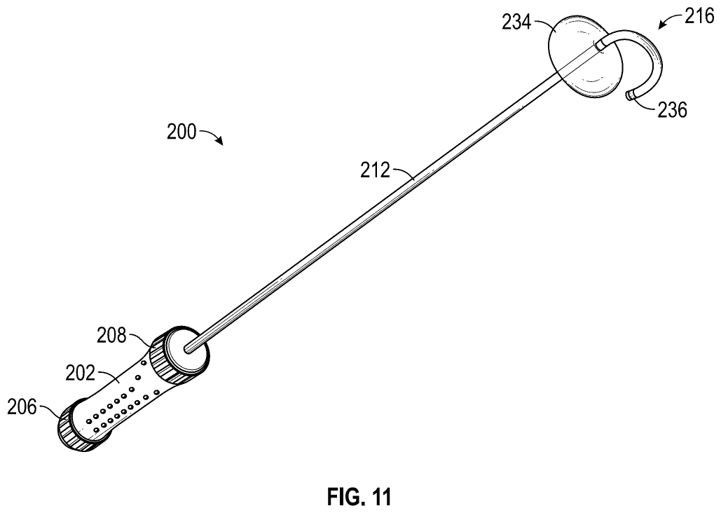

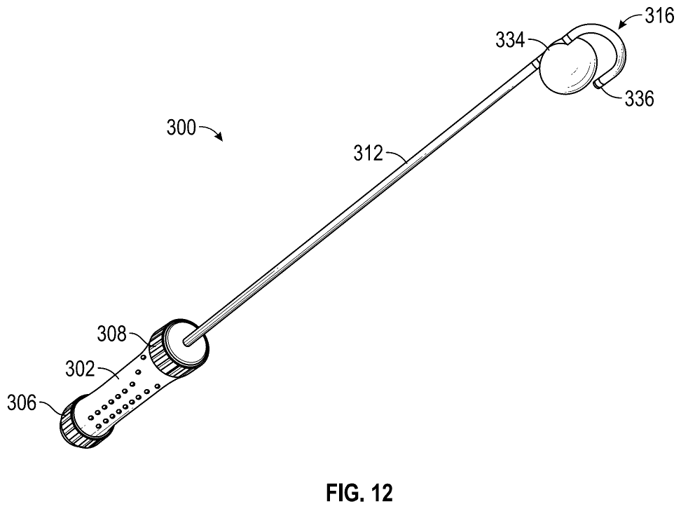

The balloon 134 may be configured in various shapes and dimensions. For example, when inflated, the offset balloon 134 may be radially symmetrical or asymmetrical with respect to a shaft axis. In one embodiment, each end of the balloon 134 may be affixed using fasteners. Alternatively, the balloon 134 may be affixed to the shaft tip 114 with an adhesive or with heat-shrinking material. In an embodiment, the balloon 134 may be inflated to an intermediate volume. For example, the balloon 134 may have a minimum deflated volume (e.g., 0 ml) and a maximum inflated volume (e.g., 10 ml). Accordingly, the balloon 134 may be configurable in the minimum deflated volume, the maximum inflated volume, or any volume in between such values. As a non-limiting example, intermediate volumes may be useful for different anatomies and different angles of distal articulation. In an embodiment, for example the embodiment illustrated in FIG. 12, the balloon 334 may be a symmetrical balloon (e.g., a round balloon with a shaft 312 positioned along the balloon's central axis). In an embodiment, such as FIGS. 7 and 11, where the balloon 134, 234 is asymmetrical, at the minimum deflated volume, the balloon 134 may exhibit no offset as the deflated balloon 134 may cling tightly to the wall of the shaft 104.

As shown in FIG. 7, the shaft tip 114 may be configured to flex in multiple dimensions. For example, the distal articulation section 116 may be configured to direct the shaft tip 114 in the z axis, and the proximal articulation section 118 may be configured to direct the shaft tip 114 in the x axis. Accordingly, the device 100 may be configured for navigating inside the pericardium via bi-directional steering in orthogonal planes.

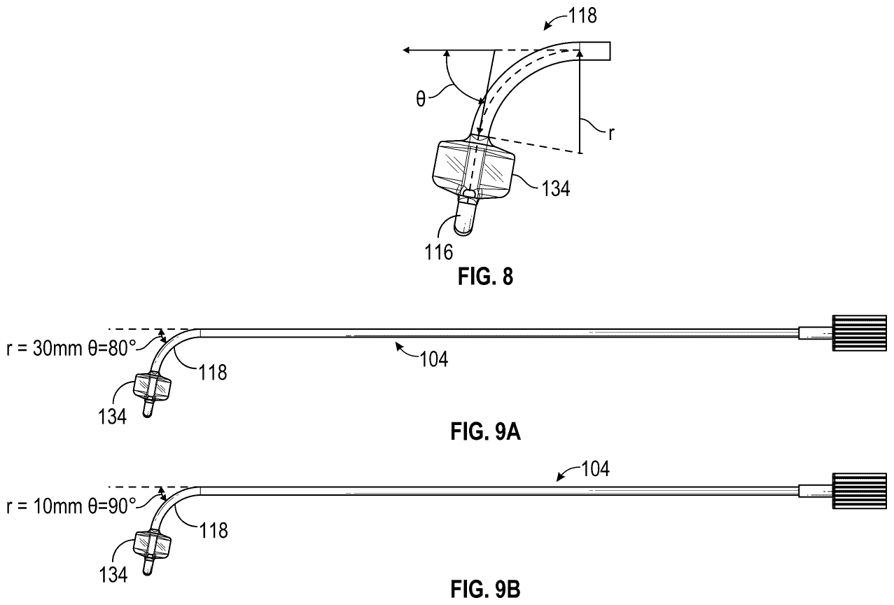

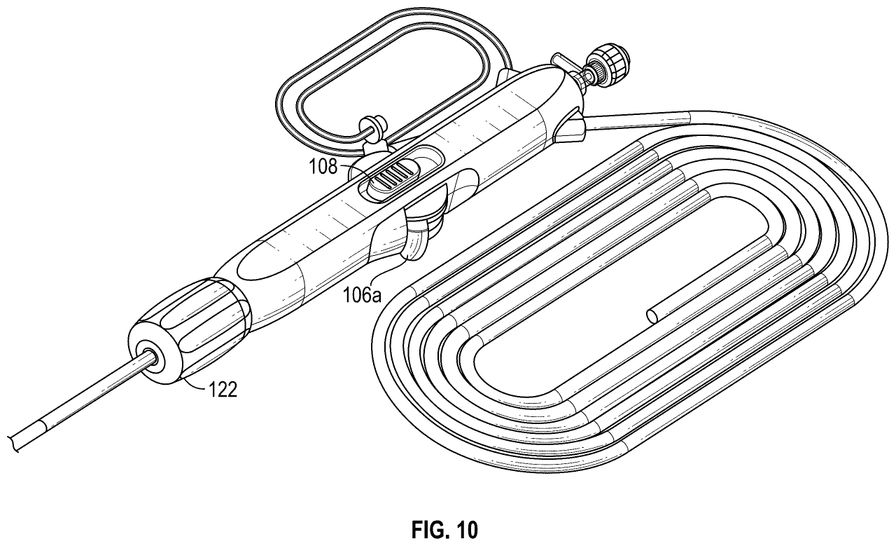

Referring to FIG. 8, the proximal articulation section 118 may be adapted to bend with a radius r with a deflection angle θ. FIG. 9A depicts an embodiment of the shaft 104, wherein the proximal articulation section 118 includes a bend radius of 30 mm and a deflection angle of 80 degrees. FIG. 9B depicts an embodiment of the shaft 104, wherein the proximal articulation section 118 includes a bend radius of 10 mm and a deflection angle of 90 degrees. Accordingly, the deflection angles of the proximal articulation section 118 and/or the distal articulation section 116 are selectable based on the tension applied to the corresponding proximal and distal articulation cables 126, 110. Therefore, the degree of actuation of the distal articulation spindle 106 and/or the proximal articulation knob 122 may induce a desired deflection angle in the distal articulation section 116 and/or the proximal articulation section 118, respectively. It should be understood that while FIGS. 9A and 9B illustrate two examples of deflection angles, any deflection angle may be utilized. For example, between −180° and 180°, −90° and 90°, −45° and 90°, or even 0° and 90°.

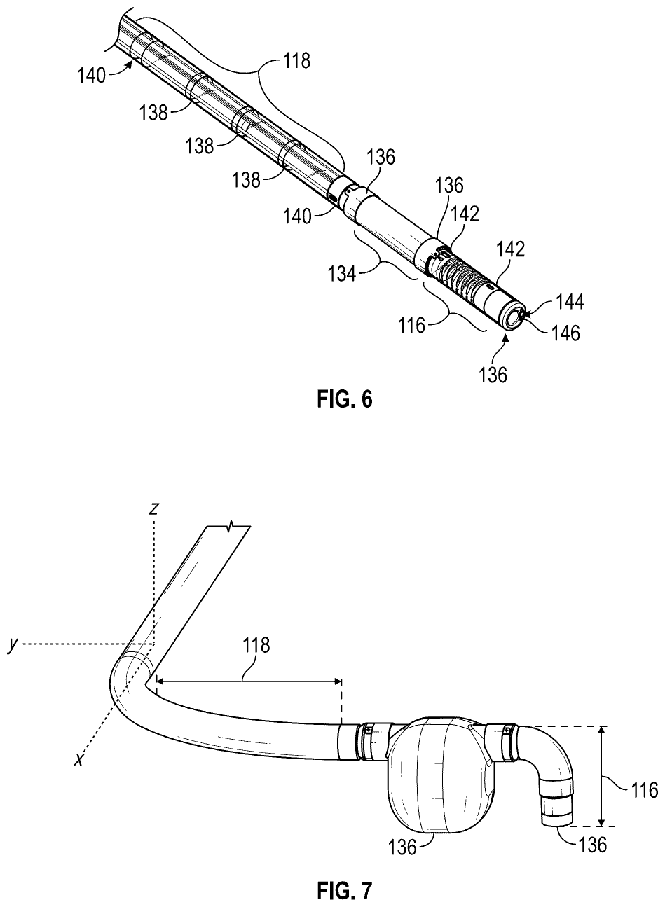

In an embodiment, as illustrated in FIGS. 5 and 6, the camera 144 may be located at the distal end of the device 100 path, for example, so that the operator may more easily visualize the working area without direct access. In a further embodiment, the device 100 includes a light source located at the tip of the device 100 path. In one such embodiment, the light source may be a light fiber bundle 146. In another embodiment, the light source may be an LED or other light emitting component disposed in the most distal portion of the shaft tip 114. In such an embodiment, the LED and power source may be entirely contained within the shaft tip 114. However, in another embodiment, the LED may be maintained within the shaft tip 114 and the power source may be external, for example, disposed within the handle 102 or external to the handle 102. In such an embodiment, a wire may traverse the shaft 104, allowing electrical communication between the LED and the power source.

In a further embodiment, the device 100 may include a transparent shield disposed on the distal end of the device 100. The transparent shield may be configured to separate the camera 144 from the tissue or other bodily structures, while permitting the camera 144 to capture images through such a transparent shield. In an embodiment, the tip may be fabricated from an optically clear material, such that the camera 144 may be recessed from the most distal tip, allowing a viewer to see the wall of a tissue even if the most distal tip is in direct contact with such tissue. As a non-limiting example, the tip could be flat or rounded and snub-nosed. This would facilitate the “tunneling” discussed herein when the device is used to traverse from the skin to the pericardium, having to navigate organs and tissues along the way.

The invention of the present disclosure may be a device 100 configured to enable navigation within the pericardium and/or visualization of access through the pericardium. Such a device 100 may comprise a device 100 path through which to pass needles or other means of puncturing the pericardium; a camera located at the distal end of the device 100 path in order to see the working area without direct access; a light source located at the tip of the device 100 path; and/or a transparent shield at the distal end of the device 100 that separates the camera 144 from the tissue but allows the camera 144 to see through it.

The device 100 may be configured to puncture through membranes and tissues (i.e., the pericardium), such that the puncture hole allows access through the pericardium. In such an embodiment, the device 100 may be configured to dilate such an access puncture. In further embodiments, the device 100 may be coupled with and/or may include a component configured for puncturing membranes and tissues, such as the pericardium. For the purposes of this disclosure, “tunneling” may refer to traversing from the skin to the outside of the pericardium. However, the device 100 may be configurable for many use cases, including, but not limited to, tunneling and/or navigation into and around various anatomical objects.

In an embodiment, the device 100 may be used to tunnel before reaching the pericardium (or other anatomical object) and/or access the pericardium (or other anatomical object). For example, first, the device 100 may be used to “tunnel,” essentially navigating around the tissue between the sternum and pericardium (i.e., lungs, diaphragm, liver, etc.) to reach the pericardium without puncturing or damaging these tissues. This procedure can be quite dangerous, and with conventional methods fragile tissue injuries may occur at this stage. In essence, conventional methods may include tunneling with a sharp needle, wherein extreme care must be exercised to avoid damaging fragile tissues on the way to the pericardium (or other target anatomical object). In an embodiment, the present disclosure contemplates a blunt-tipped obturator or dilator through the working channel of the device 100, wherein tunneling may occur under direct video observation, obtained from the camera 144, to decrease the chances of injuring fragile tissues. Next, the pericardium itself may be punctured and may be dilated to a size that a portion of the device 100 can pass through. Therefore, in passing through such fragile areas, if the device 100 enables visualization, the procedure may be made substantially safer.

FIG. 11 illustrates another embodiment of a device 200 composed of two major components, such as a handle 202 and a shaft 212. The handle 202 may be configured to be gripped by the user, wherein such manipulation induces one or more actions in shaft 212. The shaft 212 may at least partially comprise a guide tube. The device 200 may be utilized in conjunction with various medical instruments (i.e., an ablation catheter) to provide access to a desired region of the body. Thus, the device 200 may be adapted with various inputs configured to accept fluids and/or tools.

In one embodiment, the shaft 212 may be deflectable, permitting the shaft 212 to be manipulated to navigate inside the pericardium. The handle 202 may comprise an internal lumen carried by the shaft 212 that houses at least a portion of the proximal end of the shaft 212 therein. The handle 202 may be sized to be conveniently held by the user and may be sized to introduce the shaft 212 into an interior body region that has been targeted for treatment. In one embodiment, the handle 202 may be constructed, for example, from molded plastic. Of course, other materials and types of manufacturing are contemplated and known to persons of ordinary skill in the art.

The handle 202 may comprise a first actuator 206 and second actuator 208 comprising a dial and internal components that can be rotationally manipulated in opposing directions. As shown in FIG. 11, the first and second actuators 206, 208 are disposed at opposing ends of the handle 202, either in front of or behind a medial portion of the handle 202. The first and second actuators 206, 208 may be coupled to a steering mechanism such that together the first and second actuators 206, 208 and the steering mechanism components comprise a steering assembly. In one aspect, the steering mechanism comprises a first set of articulation cables, for example one or more tensioning lines, disposed within the handle 202 and comprise, for example, a cord, a Kevlar line, a control wire, or other line made from metallic or non-metallic materials. The first set of articulation cables may be coupled to the handle 202 such that a tension force (e.g., pulling) acting on the one or more tensioning lines operates to flex the shaft 212. In one embodiment, the one or more tensioning lines may comprise at least a first tensioning line and a second tensioning line, however, any number of tensioning lines are contemplated

In some embodiments, the one or more tensioning lines travel substantially along an entire length of the shaft 212 to the distal end portion 216 to couple with a steering mechanism inside the handle 202. More particularly, in an embodiment, the tensioning lines may travel within a secondary lumen or another dedicated lumen through the shaft 212 when outside the handle 202. The one or more tensioning lines may terminate at a termination point within the shaft 212 and may comprise any of the characteristics of termination points discussed herein.

In one embodiment, the guide tube may be at least partially disposed of within the handle 202. In another embodiment, the guide tube may be disposed of at least partially outside of the handle 202. It is contemplated that a portion of the guide tube may be disposed of within a sidewall of the handle 202 as the guide tube transitions from outside the handle 202 to inside the handle 202.

In one embodiment, the guide tube comprises an exterior diameter that is greater when outside of handle 202 than the exterior diameter of the guide tube inside handle 202, while the interior diameter of the guide tube remains the same regardless. Of course, in other embodiments, the exterior diameter of the guide tube may be the same regardless of its position.

The shaft 212 may be constructed, for example, by extrusion using standard flexible, medical grade plastic materials or by other means and other materials as is known in the art. It is contemplated that the shaft 212, while flexible, may comprise a plastic memory or bias that normally orients the distal end portion 216, interchangeably referred to as the distal end portion 216 of the shaft 212 or the distal articulation region in any desired configuration. For example, in a neutral state, the distal end portion 216 may be in an essentially straight configuration or in a curved configuration.

In an embodiment, the steering mechanism may be actuated by a first set of actuators, comprising the first and second actuators 206, 208, to deflect the distal end portion 216 of shaft 212 out of its neutral configuration and into a bent or deflected configuration. The change in configuration may be accomplished by a first set of articulation cables coupled to shaft 212 at the distal end portion 216 and may be tensioned by the steering mechanism to provide a force that deflects the distal end portion 216 of the shaft 212. It is contemplated that the steering mechanism may enable greater control of the orientation of the distal end portion 216 even if a plastic memory or bias is used to resist a deflected distal region. For example, in one embodiment, once deflected to a desired configuration, first and second actuators 206, 208 may be configured to be locked in place to maintain the distal end portion 216 of the shaft 212 in a desired configuration.

It is contemplated that maintaining the deflected configuration may maintain a treating tool placed within the shaft 212 in a desired relationship during use. For example, an ablation catheter, or other treating tool, may be maintained within the shaft 212 and may be positioned in a desired position within an anatomical structure by deflection of the shaft 212. In some embodiments, the steerable shaft 212 may reduce, or even obviate, the need to equip the treating tool with an on-board steering mechanism or a guide wire lumen. In some embodiments, each of the first and second actuators 206, 208 control the first set of articulation cables. For example, each of the first and second actuators 206, 208 may control one or more tensioning lines. The clinician can therefore move the distal end portion 216 of shaft 212 in the same plane of movement from either the distal or proximal end of the handle 202. It is contemplated this may optimize the operator's ability to advance/operate the handle 202 with either hand. A person of ordinary skill in the art will recognize single-handed operation of the surgical device may be particularly useful when advancing a treatment catheter or operating a treatment tool with one hand while holding and/or operating the device 200 with the other hand.

In one embodiment, the steering mechanism comprises the first and second actuators 206, 208. In some embodiments, each of the actuators may comprise internal teeth that are adapted to meet with teeth on a gear disposed about a linear drive shaft that is configured for rotation and translation relative to the first and second actuators 206, 208. In an embodiment, the drive shaft may comprise a first set of spiraled threads with a pitch oriented in a first direction and a second set of spiraled threads having a pitch oriented in a second direction and a shaft hub disposed in the center of the shaft and between the first and second set of spiraled threads. A first drive, also referred to herein as a drive nut, and second drive may be disposed about opposing sides of the linear drive shaft. Each of the first and second drive nuts comprise an aperture within the main body with internal threads adapted to mate with the threads of the shaft. In some embodiments, the drive nuts further comprise a tab or wing portion extending away from the main body such that wings of the respective drive nuts are on opposing sides of the shaft inside handle. In an embodiment, the drive nuts may be configured to mate with the one or more tensioning lines that also extend longitudinally about opposing sides of the shaft 212 to the distal end portion 216. In one embodiment, the tensioning lines are coupled to apertures located within the wing of drive nuts however, the one or more tensioning lines may be soldered, bonded, or otherwise coupled to the drive nuts so long as the linear movement of the nuts translates into linear movement of the tensioning lines.

As the first actuator 206 and/or second actuator 208 is rotated, the gear may be likewise rotated resulting in rotation of the linear drive shaft. The rotational movement of the linear drive shaft may translate into linear movement of the drive nuts about drive shaft. In one embodiment, the respective pitches of the first and second spiraled threads are substantially the same even though they are oriented in different directions. For example, as the drive shaft is rotated, drive nuts will travel the same linear distance about the shaft 212. However, because the direction of the pitch of the first and second threads are oriented in opposite directions (i.e., one is a left-handed thread while the other is a right-handed thread) the direction of the linear travel of the respective drive nuts will be in opposite, but parallel directions. Consequently, a tensioning force will act upon the one or more tensioning lines respectively depending on the direction of rotation of the first actuator 206 and/or second actuator 208. For example, when either of the first or second actuators 206, 208 are rotated about the handle, the drive nut may move linear to the proximal end of the handle 202 resulting in a tensioning force acting on the one or more tensioning line.

In one embodiment, first and second actuators 206, 208 are configured to operate the first linear drive shaft and the spindle. In one embodiment, the first and second actuators 206, 208 comprise a plurality of internal teeth that mate with the teeth of a gear of the drive shaft. When either of the first and second actuators 206, 208 is rotated, the drive shaft may also be rotated. In one embodiment, the spindle also comprises a gear with teeth configured to mate with the first and second actuators 206, 208. However, in another embodiment, the gears of the spindle may be longitudinally offset from the first and second actuators 206, 208. That is, in some embodiments, the gears associated with the spindle are slightly closer to the center of the spindle than the gears located on the first drive shaft. The first and second actuators 206, 208 may be disposed about the handle 202 such that they are longitudinally biased to engage the gear of the drive shaft.

A person of ordinary skill in the art will recognize that a number of different mechanisms may be employed to create tension in the one or more tensioning lines without departing from the scope of the disclosed technology, any of which may be utilized to carry out the device. For example, in one embodiment, the one or more tensioning lines may comprise a shape memory alloy (e.g., Nitinol) that is biased in a straight or linear configuration but when subjected to an electrical signal or charge, will flex into a pre-determined configuration. In this aspect, the second actuator 208 disposed on a proximal end of the handle 202 would function to flex the distal end portion 216 of the shaft 212 in both the negative and positive directions of a plane. Likewise, the first actuator 206 disposed on a distal end of the handle 202 would operate the same tensioning lines associated with the second actuator and function to flex the distal end portion 216 of shaft 212 in positive and negative directions on a plane.

In accordance with one embodiment, a spindle is enclosed within the handle 202 and coupled to a second set of actuators. The spindle may be coupled to third and fourth drive nuts that operate to create a tensioning force in a second set of articulation cables, comprising a third and fourth tensioning lines.

In one embodiment, the third and fourth drive nuts are offset from the first and second drive nuts about the circumference of the handle 202 by 90 degrees. It is contemplated that the third and fourth tensioning lines may extend longitudinally about the length of the shaft 212 through secondary lumens. In an embodiment, movement of the third and fourth drive nuts result in movement of the third and fourth tensioning lines which function to flex the distal end portion 216 of the shaft 212 in a positive or negative direction on a plane of movement. For example, while the first and second tensioning lines flex the distal end portion 216 of shaft 212 in a single plane of movement, the third and fourth tensioning lines function to flex the distal end portion 216 of the shaft 212 in an additional plane of movement. While a 90 degree offset has been referenced herein, it is understood that the third and fourth tensioning lines can be oriented to deflect the distal end portion 216 of the shaft 212 in any plane offset from the plane of operation associated with the first and second tensioning lines. For example, in one aspect of the technology, the third and fourth tensioning lines are offset from the plane of operation associated with the first and second tensioning lines by 15 degrees, 30 degrees, 45 degrees, or 60 degrees. The third and fourth tensioning lines may also be offset at other orientations that lie between 1 and 89 degrees as suits a particular application.

In accordance with one aspect of the technology, similar to the first linear drive shaft, the spindle comprises threads oriented in opposing directions. Likewise, the opposing threads have a similar pitch so that the distance of linear travel of the associated drive nuts is equivalent even though the direction of the travel is in opposite directions. However, in another embodiment, the threads of the spindle have a pitch having a lighter or less angled pitch than the pitch of the threads on the first linear drive shaft. A lighter or shallower pitch results in smaller linear travel with the same amount of rotation of the drive shaft. In some embodiments, the clinician can place the distal end portion 216 of the shaft 212 in a “rough” location by using the first set of actuators and the first linear drive shaft and switch to the second set of actuators and the spindle to flex the distal end portion 216 of shaft at smaller intervals for more precise movements. In some embodiments, the third and fourth tensioning lines can terminate at the same longitudinal location as the first and second tensioning lines.

In another embodiment, the second set of actuators may induce the proximal articulation deflection angle in a proximal articulation section of the shaft. Manipulation of the second set of actuators may result in deflection of the second set of articulation cables. For example, the second set of articulation cables may be deflected in accordance with any manner described herein. In one embodiment, the balloon may be disposed of between the distal and proximal articulation sections. However, the balloon may be positioned at any point along the shaft.

Returning to the embodiment illustrated in FIG. 11, the proximal end of the handle 202 comprises hemostasis or backflow check seals or valves. It is contemplated that the seals or valves may reduce blood loss and retrograde flow of air into the circulatory system. In an embodiment, a hub may be disposed of on the proximal end of the handle 202 and may comprise the hemostasis seal. In some embodiments, the seal may comprise an annular soft elastomeric gasket configured to seal against catheters, instruments, and the dilator, inserted therethrough. In one embodiment, the seal may further comprise a valve such as a stopcock, one-way valve such as a duckbill or flap valve, or the like to prevent significant blood loss and air entry when an instrument or catheter is removed from the lumen of the device 200. In a further embodiment, the soft annular seal may comprise a mechanism to compress the inner diameter of the seal radially inward, such as the mechanisms found on Tuohy-Borst valves.

In one embodiment, the hub comprises one or more sideports for injection of contrast media such as Omnipaque, Renografin, or other Barium-loaded solutions, for example. In an embodiment, the one or more sideports may be used for the injection of anticoagulant solutions such as heparin, coumadin, persantin, or the like. In a further embodiment, the one or more sideports may be used for the measurement of pressure at or near the distal end portion 216 of the device 200. Of course, in other embodiments, fluids may be injected directly through the valve in the hub.

In one embodiment, a plurality of hemostasis valves and/or fluid input connectors or ports are disposed about the hub. In an embodiment, the hub comprises a central lumen coupled directly to a Tuohy-Borst valve integrally formed with the hub. In some embodiments, the lumen of the hub may be coupled to the lumen of the handle 202. In other embodiments, the lumen of the hub may comprise the proximal end of the handle 202 itself. In one embodiment, a hemostasis adapter may be inserted through the Tuohy-Borst valve on the hub. The Tuohy-Borst valve may be configured to tighten around the hemostasis adaptor to obtain a seal around the hemostasis adaptor tubing. It is contemplated that the Tuohy-Borst valve may be configured to seal about the tubing with a diameter ranging from 10 to 30 French. For example, the Tuohy-Borst valve may seal around a diameter form about 5 to 6 French, from about 5 to 10 French, or from about 6 to 18 French.

In one aspect, a stopcock and purge line can be used for aspiration of blood or saline or the purging of air from the hub lumen.

In some embodiments, the distal end portion 216 may comprise one or more impedance sensors 236. In one embodiment, the one or more impedance sensors 236 may be exposed metal contacts coupled to insulated electrical wires, further coupled to an electroanatomic mapping (EAM) system operative to provide positioning data via impedance, resistance, current, or other electrical signals. As a non-limiting example, the impedance sensors 236 may be any positional markers of the type used in anatomic mapping (e.g., impedance electrodes used in impedance-based anatomical mapping, etc.) and/or may be positioned across an area occupied by the monitoring element. Further, impedance sensors 236 may be used to three-dimensionally map the location of the distal end portion 216 within the patient's body. The device 200 may be configured for navigating inside the heart endocardium or outside the heart and inside the pericardium in the epicardial space, wherein the device 200 may also include exposed electrodes along the length of the shaft 212 to allow integration into cardiac impedance mapping systems. It is contemplated that such an embodiment may permit the location and orientation of the device 200 path to be illustrated on a 3D model of the heart on an accompanying display.

The most distal point of the distal end portion 216 may be referred to as a “muzzle.” The muzzle may encompass the distal face of an exposed portion of the shaft 212. It is contemplated that the muzzle may be rounded or otherwise configured to reduce drag or potential harm to bodily structures.

In one embodiment, the distal end portion 216 may further comprise a balloon 234 disposed of proximal to the muzzle. In some embodiments, the balloon 234 may be disposed of directly proximal to the impedance sensors 236. In another embodiment, the balloon 234 may be disposed of directly distal to the impedance sensors 236. In still a further embodiment, the balloon 234 may be disposed of proximal to a first impedance sensor and distal to a second impedance sensor.

The balloon 234 may be inflated to create space between tissue, vessel, or other anatomical features and the distal end portion 216. It is contemplated that the space created by the balloon 234 in the inflated state may permit movement and rotation of the distal end portion 216. For example, the inflated balloon 234 may be positioned against tissue, vessel, or other anatomical features, wherein the balloon 234 is configured to provide stability to the distal end portion 216. In another non-limiting example, the balloon 234 may be configured and/or utilized to push anatomical structures away from the distal end portion 216 for the protection of said anatomical structures.

In one embodiment, the balloon 234 may be inflated with a liquid and/or a gas. As a non-limiting example, the balloon 234 may be inflated with air, saline, water, a saline-water mixture, a contrast agent, and/or a combination thereof. The balloon 234 chamber may be in fluid communication with a fluid source, for example, exterior to the shaft 212 and handle 202. In an embodiment, a fluid lumen may be disposed along the handle 202 and/or the shaft 212, such that fluid may be introduced to and/or removed from the balloon 234. For example, a separate inflation tubing may run parallel within the internal lumen along the inside of the shaft 212. Thus, in one embodiment, the fluid lumen may be a singular lumen configured to both introduce and remove fluid. In another embodiment, the fluid lumen may be a multi-lumen, comprising two or more lumens, each of the two or more lumens configured for exclusive removal or introduction of fluid.

In one embodiment, each end of the balloon 234 may be affixed to the shaft 212 using fasteners. In another embodiment, the balloon 234 may be affixed to the shaft 212 with an adhesive or with heat-shrinking material. Of course, any manner of affixing the balloon 234 to the shaft 212 is contemplated and may be utilized.

Referring to the balloon 234 illustrated in FIG. 11, in some embodiments the balloon 234 may be a symmetric balloon (e.g., a round balloon with the shaft 212 positioned along the balloon's central axis). However, as shown in FIG. 12, in other embodiments the balloon 334 may be an asymmetric balloon.

In an embodiment, such as the embodiment illustrated in FIG. 11, the balloon 234 may be inflated to an intermediate volume. For example, the balloon 234 may have a minimum deflated volume (e.g., 0 ml) and a maximum inflated volume (e.g., 10 ml). Accordingly, the balloon 234 may be configurable in the minimum deflated volume, the maximum inflated volume, or any volume in between such values. As a non-limiting example, intermediate volumes may be useful for different anatomies and different angles of distal articulation. In an embodiment where a balloon 334 is asymmetrical, such as the embodiment illustrated in FIG. 12, at the minimum deflated volume, the balloon 334 may exhibit no offset as the deflated balloon 334 may cling tightly to the wall of the shaft 104.

Referring without limitation to FIG. 11, in an embodiment, the device 200 may be used to tunnel before reaching the pericardium and/or access the pericardium (or other anatomical object). For example, first, the device 200 may be used to “tunnel,” essentially navigating around the tissue between the sternum and pericardium (i.e., lungs, diaphragm, liver, etc.) to reach the pericardium without puncturing or damaging these tissues. In other embodiments, the device 200 may be used in relation to other anatomical structures, such as lungs, diaphragm, liver, and any other anatomical structure a person of ordinary skill in the art may desire.

FIG. 12 further illustrates a device 300 comprising a handle 302, a first actuator 306, a second actuator 308, and a shaft 312 having a distal end 316 and the asymmetric balloon 334. The shaft 312 may comprise one or more impedance sensors 336. For example, as illustrated the one or more impedance sensors 336 may be disposed of at or near the distal end 316 of the shaft 312. The device 300 illustrated in FIG. 12 may comprise any of the characteristics discussed in detail herein.

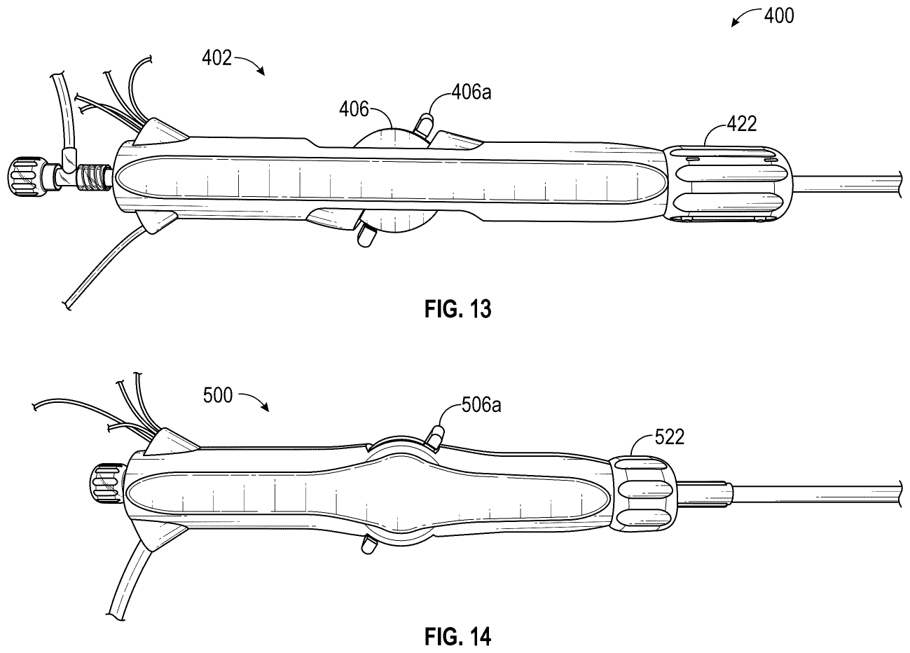

Another embodiment of a handle 402 for a device 400 is illustrated in FIG. 13. The handle 402 may comprise a first actuator and a second actuator. The first actuator may comprise a lever 406a and a distal articulation spindle 406. As illustrated, the second actuator may be a proximal articulation control knob 422. The first and second actuators described in reference to FIG. 13 may comprise any of the characteristics discussed herein. For example, any of the characteristics discussed with reference to FIGS. 1-4. However, any actuators may be utilized and the aforementioned are provided as non-limiting examples.

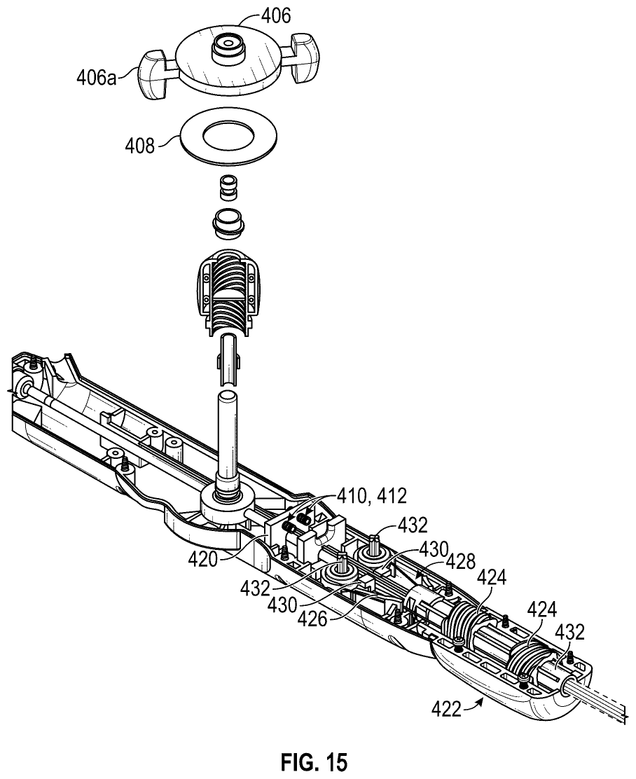

FIG. 15 illustrates a sectional view of one embodiment of the handle 402. In the embodiment illustrated, the distal articulation spindle 406 may be in frictional contact with a disc 408. The disc 408 may be operative to increase friction at the distal articulation spindle 406, preventing the distal articulation spindle 406 from rotating without manipulation.

In an embodiment, the disc 408 may be a silicone disc and the distal articulation spindle 406 may comprise acrylonitrile butadiene styrene (ABS) plastic. It is contemplated that the silicone and ABS plastic may be used in conjunction to create a high-friction interface, such that the distal articulation spindle 406 will be locked in place until an external force acts on the distal articulation spindle 406, for example by rotating the lever 406a. Other materials may be utilized for the disc 408 and/or the distal articulation spindle 406 and the aforementioned are provided as examples. As a nonlimiting example, the arrangement of the disc 408 induces a consistent friction perceivable by the user during actuation of lever 406a. Yet further, the friction imposed between the aforementioned components permits the distal articulation angle to remain at the angle last selected by the user via the lever 406a. Thus, the frictional relationship between the disc 408 and adjacent components permits a passive locking schema, enabling manipulation of the device with less switching and clicking by the user. Such improvements may be visible over embodiments adorned with separate switches and levers configured solely for locking the angle of distal articulation.

As illustrated in FIG. 15, the handle 402 may comprise one or more distal articulation cables 410, distal articulation cable housings 412, a distal articulation tension adjustment 420, a plurality of proximal articulation drive nuts 424, one or more proximal articulation cables 426, proximal articulation cable housings 428, s proximal articulation tension adjustment 430, and/or proximal articulation pulleys 432. Any of the components of the handle 402 may comprise any of the characteristics discussed in reference to FIGS. 1-4.

FIG. 14 illustrates another embodiment of a handle 500 comprising a first actuator, comprising a lever 506a and a spindle, and a second actuator, illustrated as a proximal articulation control knob 522. The proximal articulation control knob 522 may, as illustrated in FIG. 14, be a short knob disposed at the end of the handle 500 adjacent to the shaft. The proximal articulation control knob 522 may comprise a plurality of recesses disposed on its outer surface to improver a user's ability to grip and rotate the proximal articulation control knob 522. However, the proximal articulation control knob 522 may be any dimension, for example, the longer proximal articulation knobs 122 and 422 illustrated in FIGS. 1 and 13. Any of the device may comprise any of the characteristics described herein. For example, the handle 500 may comprise any of the internal components, such as the disc 408, discussed with reference to FIGS. 13 and 15.

The following examples relate to non-limiting manners in which the teachings of the present disclosure may be practiced or combined. The following examples are provided for illustrative purposes. Further, variations of the following examples may omit certain features. Accordingly, none of the components or features depicted below should be viewed as critical unless explicitly indicated.

Example 1: A medical device comprising a handle comprising a first actuator mechanically coupled to a first set of articulation cables, and a second actuator; a shaft coupled to the handle, the shaft comprising a distal articulation section configured to bend with a distal articulation deflection angle and a distal articulation radius, wherein the distal articulation section is mechanically coupled to the first set of articulation cables, and wherein actuation of the first actuator is configured to induce the distal articulation deflection angle in the distal articulation section; and a balloon disposed of adjacent to the distal articulation section of the shaft, wherein the balloon is configurable in a deflated state and an inflated state.

Example 2: The medical device of example 1, wherein the shaft further comprises a proximal articulation section configured to bend with a proximal articulation deflection angle and a proximal articulation radius, wherein the proximal articulation section is mechanically coupled to a second set of articulation cables.

Example 3: The medical device of example 2, wherein actuation of a proximal articulation control knob is configured to induce the proximal articulation deflection angle in the proximal articulation section.

Example 4: The medical device of example 1, further comprising a light fiber bundle disposed in the shaft, traversing from at least the handle to the distal articulation section.

Example 5: The medical device of example 1, further comprising a light source disposed within the distal articulation section.

Example 6: The medical device of example 1, wherein actuation of the first actuator is configured to bend the shaft in a first plane, and actuation of the second actuator is configured to bend the shaft in a second plane.

Example 7: The medical device of example 6, wherein the first plane and the second plane are perpendicular.

Example 8: The medical device of example 1, wherein the balloon is asymmetric around the shaft.

Example 9: The medical device of example 1, wherein the balloon is symmetric around the shaft.

Example 10: The medical device of example 1, further comprising one or more impedance sensors disposed of at the distal articulation section of the shaft.

Example 11: The medical device of example 10, wherein any of the one or more impedance sensors are disposed distally to the balloon.

Example 12: The medical device of example 10, wherein any of the one or more impedance sensors are disposed proximally to the balloon.

Example 13: The medical device of example 1, the handle further comprising a first articulation lock in mechanical communication with the first actuator, the first articulation lock configured to lock the distal articulation section in a desired position.

Example 14: The medical device of example 1, the handle further comprising one or more proximal articulation drive nuts in mechanical communication with a second set of articulation cables.

Example 15: The medical device of example 14, the handle further comprising one or more proximal articulation pulleys in mechanical communication with the second set of articulation cables.

Example 16: The medical device of example 1, wherein the first and second actuators are both coupled to a first drive adapted to pull on the first set of articulation cables and a spindle adapted to pull on a second set of articulation cables, and wherein the first drive and the spindle are disposed about a linear drive shaft.

Example 17: The medical device of example 1, further comprising a camera disposed on the shaft.

Example 18: The medical device of example 1, further comprising a disc in frictional contact with the first actuator to prevent the first actuator from rotating without manipulation.

Various elements, which are described herein in the context of one or more embodiments, may be provided separately or in any suitable subcombination. Further, the processes described herein are not limited to the specific embodiments described. For example, the processes described herein are not limited to the specific processing order described herein and, rather, process blocks may be re-ordered, combined, removed, or performed in parallel or in serial, as necessary, to achieve the results set forth herein.

It will be further understood that various changes in the details, materials, and arrangements of the parts that have been described and illustrated herein may be made by those skilled in the art without departing from the scope of the following claims.

All references, patents and patent applications and publications that are cited or referred to in this application are incorporated in their entirety herein by reference. Finally, other implementations of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

What is claimed is:

1. A medical device comprising:
a handle comprising:
a first actuator mechanically coupled to a first set of articulation cables, and
a second actuator;
a shaft coupled to the handle, the shaft comprising:
a distal articulation section configured to bend with a distal articulation deflection angle and a distal articulation radius,
wherein the distal articulation section is mechanically coupled to the first set of articulation cables, and wherein actuation of the first actuator is configured to induce the distal articulation deflection angle in the distal articulation section; and
a balloon disposed of adjacent to the distal articulation section of the shaft, wherein the balloon is configurable in a deflated state and an inflated state.

2. The medical device of claim 1, wherein the shaft further comprises:
a proximal articulation section configured to bend with a proximal articulation deflection angle and a proximal articulation radius,
wherein the proximal articulation section is mechanically coupled to a second set of articulation cables.

3. The medical device of claim 2, wherein actuation of a proximal articulation control knob is configured to induce the proximal articulation deflection angle in the proximal articulation section.

4. The medical device of claim 1, further comprising a light fiber bundle disposed in the shaft, traversing from at least the handle to the distal articulation section.

5. The medical device of claim 1, further comprising a light source disposed within the distal articulation section.

6. The medical device of claim 1, wherein actuation of the first actuator is configured to bend the shaft in a first plane, and actuation of the second actuator is configured to bend the shaft in a second plane.

7. The medical device of claim 6, wherein the first plane and the second plane are perpendicular.

8. The medical device of claim 1, wherein the balloon is asymmetric around the shaft.

9. The medical device of claim 1, wherein the balloon is symmetric around the shaft.

10. The medical device of claim 1, further comprising one or more impedance sensors disposed of at the distal articulation section of the shaft.

11. The medical device of claim 10, wherein any of the one or more impedance sensors are disposed distally to the balloon.

12. The medical device of claim 10, wherein any of the one or more impedance sensors are disposed proximally to the balloon.

13. The medical device of claim 1, the handle further comprising a first articulation lock in mechanical communication with the first actuator, the first articulation lock configured to lock the distal articulation section in a desired position.

14. The medical device of claim 1, the handle further comprising one or more proximal articulation drive nuts in mechanical communication with a second set of articulation cables.

15. The medical device of claim 14, the handle further comprising one or more proximal articulation pulleys in mechanical communication with the second set of articulation cables.

16. The medical device of claim 1, wherein the first and second actuators are both coupled to a first drive adapted to pull on the first set of articulation cables and a spindle adapted to pull on a second set of articulation cables, and wherein the first drive and the spindle are disposed about a linear drive shaft.

17. The medical device of claim 1, further comprising a camera disposed on the shaft.

18. The medical device of claim 1, further comprising a disc in frictional contact with the first actuator to prevent the first actuator from rotating without manipulation.
